(19) United States
(12) Patent Application Publication
(10) Pub. No.: US 2012/0059311 A1
(43) Pub. Date: Mar. 8, 2012

(54) PORTABLE THERAPEUTIC GAS DISPENSING DEVICE

(75) Inventor: Scott Jay GILBERT, Menlo Park, CA (US)

(73) Assignee: Capnia, Inc., Palo Alto, CA (US)

(21) Appl. No.: 13/039,713

(22) Filed: Mar. 3, 2011

Related U.S. Application Data
(60) Provisional application No. 61/310,117, filed on Mar. 3, 2010.

(51) Int. Cl. A61M 37/00

(52) U.S. Cl. 604/26

(57) ABSTRACT

Described here are hand-held dispensers for intranasally delivering a therapeutic gas such as carbon dioxide to a user. The dispensers generally include a compressed gas cylinder, a pierce pin block, a valve, a regulator tube and a nosepiece. The regulator tube regulates both the pressure and flow of the gas out of the dispenser.

### CROSS-REFERENCE TO RELATED APPLICATIONS

This application claims priority to U.S. Provisional Application Ser. No. 61/310,117, filed on Mar. 3, 2010, which is hereby incorporated by reference in its entirety.

### FIELD

Described here are portable, hand-held devices for dispensing and administration of a therapeutic gas to the nasal mucosa of a user, e.g., a patient. Specifically, hand-held devices that include a regulator tube for controlling the flow rate and pressure of a therapeutic gas are described. Methods for regulating gas flow and pressure from the hand-held dispenser for safe and controlled intranasal delivery of a pressurized therapeutic gas are also described.

### BACKGROUND

A typical compressed gas pressure regulator incorporates a spring-loaded diaphragm mechanism that regulates the opening and closing of a gas discharge orifice. This mechanism can be calibrated manually to provide constant delivery pressure at any value within a designated range. After the desired delivery pressure is set, the regulator opens or closes the gas discharge to maintain constant pressure. In turn, the flow rate is controlled by the use of a separate restricting orifice or similar component. The pressure regulating and flow rate controlling components are widely known and practiced in the art. Pressure regulators, however, are typically bulky and expensive, making them less than ideal for use in a compact, hand-held, low-cost dispensing device. Consequently, it would be beneficial to have a simple alternative for regulating the dispensing pressure and flow rate of a compressed gas that met the objectives of compactness, low-cost, reliability and ease of manufacture.

### SUMMARY

Described here are regulator tubes for regulating the flow and pressure of a therapeutic gas for intranasal delivery from a portable, hand-held device. That is, a single element (the regulator tube) regulates both the pressure and flow rate. The combination of both aspects in a single element (e.g., instead of using a pressure regulator for controlling pressure and a limiting orifice for controlling flow), may help to provide an intranasal gas delivery device that is compact, and which can be manufactured at a low-cost, easily and reliably. As used herein, the terms “tube” and “regulator tube” are used interchangeably. “Regulator tube” is used in order to indicate the flow and pressure regulation aspect of the tube. Exemplary hand-held devices in which the regulator tubes described here may be beneficial include those described in U.S. Pat. No. 7,845,347, which is co-owned by the assignee, and which is hereby incorporated by reference.

The hand-held dispensers described herein generally include a regulator tube having an inlet end, an outlet end, and a length therebetween, and further, an internal diameter. The hand-held dispensers also generally include a compressed gas cylinder comprising a therapeutic gas, a valve that couples the gas cylinder to the inlet end of the regulator tube, and a nosepiece coupled to the outlet end of the regulator tube. The valve may be a stem valve. Any suitable gas may be delivered by the hand-held devices. For example, carbon dioxide may be delivered.

Methods for intranasally delivering a therapeutic gas to a user are also described herein. In general, the method includes inserting a hand-held dispenser including a regulator tube into a nostril of a user, and actuating a valve to selectively discharge the therapeutic gas from the compressed gas cylinder through the regulator tube and the nosepiece. The regulator tube typically includes an inlet end, an outlet end, and a length therebetween, and also an internal diameter. The hand-held dispensers usually include a compressed gas cylinder comprising a therapeutic gas; a valve that couples the gas cylinder to the inlet end of the regulator tube; and a nosepiece coupled to the outlet end of the regulator tube. The regulator tube regulates the flow and pressure of the therapeutic gas released from the compressed gas cylinder.

The methods for delivering a therapeutic gas described above may be employed to treat various medical conditions. For example, the therapeutic gases may be delivered to treat symptoms associated with headache (e.g., migraine headaches, tension-type headaches, cluster headaches), jaw pain, facial pain (e.g., trigeminal neuralgia), allergic conditions (e.g., rhinitis, including seasonal allergic rhinitis and perennial allergic rhinitis, and conjunctivitis), asthma, nervous disorders (e.g., epilepsy, Parkinson's disease), ischemic heart disease and pulmonary hypertension. Some variations of the method employ carbon dioxide as the delivered gas to treat seasonal allergic rhinitis or perennial allergic rhinitis. In another variation, nitric oxide or its variants is delivered to treat ischemic heart disease. In yet further variations, nitric oxide or its variants is delivered to treat pulmonary hypertension.

### BRIEF DESCRIPTION OF THE DRAWINGS

FIG. 1 illustrates an exemplary hand-held dispenser including a regulator tube according to one variation.

FIG. 2 illustrates the flow rate performance for a hand-held dispenser including another variation of a regulator tube.

FIG. 3 illustrates the flow rate performance for a hand-held dispenser including a further variation of a regulator tube.

### DETAILED DESCRIPTION

Regulator tubes for regulating the gas flow and pressure from a portable, hand-held device for intranasal delivery of a therapeutic gas are described herein. The regulator tubes may replace the typical compressed gas pressure regulators that incorporate spring-loaded diaphragm mechanisms that regulate the opening and closing of a gas discharge orifice. As previously noted, conventional pressure regulators are typically bulky and expensive, making them less desirable for use in a compact, hand-held, low-cost dispensing device.

The beneficial aspects of the regulator tubes described here over conventional gas pressure regulators for a hand-held dispenser of a therapeutic gas may include:
1. No calibration is required. Pressure and flow rate may be controlled precisely based on tube length and tube diameter.
2. Pressure and flow rate regulation may be achieved through the use of a simple element (i.e., a tube).
3. Dead volume may be minimized compared to a conventional pressure regulator. Dead volume refers to the air space that a gas must fill as it transits through a regulating element. If the air space is large, then more gas is consumed per dispense compared to a small air space since the gas that fills the air space is generally wasted.
4. Low cost of manufacture/low overall part count.
5. Inherently safe since a significant pressure spike that would pose a danger to the user may not occur. Specifically, conventional regulators can potentially fail in such a way that high pressure gas can be delivered without effective regulation (e.g., the diaphragm can break or the regulator can be miscalibrated). The regulator tube may not have this type of failure mode because the tube strength is sufficient to hold the pressure of the delivered gas. Further, the gas must necessarily travel through the tube, and thus, be regulated.
6. Compactness.
7. Simple manufacture/less risk of manufacturing error or tolerance issues.
8. Narrow tube may have high burst pressure and low hoop stress (i.e., high safety factor).

The inventors have discovered that utilizing a regulator tube may be a reliable and cost effective apparatus for implementing a hand-held dispenser of a therapeutic gas to a user. By varying the length and diameter of the regulator tube, the flow rate of the therapeutic gas may be controlled. Flow through the regulator tube may be based on the Hagen-Poisseuille equation:

$$Q = \frac{π {Pd}^{4}}{128 μ l}$$

or, in terms of pressure:

$$Δ P = \frac{128 μ l Q}{π {d}^{4}}$$

Where
   Q=flow rate
   ΔP=pressure drop across tube
   d=tube diameter
   l=tube length
   μ=dynamic viscosity of the fluid

The equation describes the relationship between the tube diameter and tube length and their respective contributions to pressure drop and fluid flow rate. (The tube diameter d refers to the internal diameter of the tube.) If the tube diameter is cut in half, for example, the fluid flow will be reduced to 1/16^{th} of the original rate. If the tube length, on the other hand, is doubled, the flow rate will be halved. Consequently, for a specific pressure condition, the tube diameter and tube length define the resulting pressure drop and flow rate. Further, these two parameters, diameter and length, can be paired in a variety of ways to accomplish the same result. Equivalent performance could be achieved, for instance, by using either a small diameter tube of comparatively short length or a larger diameter tube of longer length. This permits the designer to specify the tube characteristics for a broad range of design parameters and constraints and reduces the overall number of parts to control the gas pressure and flow rate.

In general, a regulator tube may be employed as a pressure and flow rate controlling element in lieu of a conventional pressure regulator and rate controlling orifice (or equivalent). The regulator tubes described here may be configured to deliver a therapeutic gas at a flow rate ranging from about 0.10 SLPM (standard liters per minute) to about 1.00 SLPM, from about 0.20 to about 0.80 SLPM, or from about 0.30 to about 0.60 SLPM. In some variations, the regulator tubes are configured to deliver a therapeutic gas at a flow rate of about 0.50 SLPM.

With respect to initial gas pressures (inlet pressures), the regulator tubes may be configured to provide initial pressures (at the inlet end) ranging from about 300 psi (pounds per square inch) to about 1800 psi, from about 600 psi to about 1200 psi, or from about 750 psi to about 900 psi. In one variation, the initial gas pressure is about 850 psi. When using the regulator tubes described here, the outlet pressure (at the outlet end) may be about 14.7 psi (1 atmosphere), which is a pressure suitable for intranasal delivery.

Any suitable therapeutic gas may be delivered by the hand-held devices. Exemplary therapeutic gases include, without limitation, carbon dioxide, nitric oxide, oxygen, hydrogen sulfide, xenon, isocapnic mixtures of gaseous acids and helium, their variants, and mixtures thereof. The therapeutic gases may be used in a substantially pure form without other gases, active agents, or other substances that dilute the therapeutic gas or that have other biological activities. In other instances, the therapeutic gases may be combined with other gases, such as inert carrier gases, active gases, solids to form aerosols, liquid droplets to form aerosols, sprays, powders, or the like to potentiate (enhance) their effects. In some variations, the treatment gas is carbon dioxide.

In view of the above, the length of the regulator tubes may range from about 2.54 cm (1.0 inch) to about 152.4 cm (60 inches), from about 2.54 cm to about 127 cm (50 inches), from about 2.54 cm to about 101.6 cm (40 inches), from about 2.54 cm to about 76.2 cm (30 inches), from about 2.54 cm to about 50.8 cm (20 inches), from about 2.54 cm to about 25.4 cm (10 inches), or from about 2.54 cm to about 12.7 cm (5 inches). In one variation, the length of the regulator tube ranges from about 12.7 cm (5 inches) to about 38.1 cm (15 inches).

The internal diameter (ID) of the regulator tubes described here may range from about 0.00254 cm (0.001 inches) to about 0.0127 cm (0.005 inches). Some variations of the tube are about 0.00254 cm (0.001 inches) to about 0.00508 cm (0.002 inches).

Regulator tubes that are about 91.4 cm (36 inches) long and have an ID of about 0.0102 cm (0.004 inches) may be beneficial to incorporate in the hand-held intranasal delivery devices. It may also be useful to include regulator tubes that are about 12.7 cm (5 inches) long with an ID of about 0.003 cm (0.001 inches).

Alternatively, regulator tubes having a ratio of length to internal diameter ranging from about 500:1 to about 12,000:1, from about 1,000:1 to about 12,000:1, from about 5,000:1 to about 12,000:1, or from about 10,000:1 to about 12,000:1 may be used. For example, the ratio of length to internal diameter may be about 9,000:1. In some instances, the ratio of length to internal diameter is about 5,000:1. In other instances, the ratio of length to internal diameter may be greater than 12,000:1.

The regulator tubes may be made from any suitable material, so long as they can withstand the pressure of the delivered therapeutic gas. For example, the regulator tubes may be formed from a polymeric material, e.g., from fluoropolymers, polyetheretherketone (PEEK), polyethylene, polyethylene terephthalate, silicone, polyamides, Pebax® polyether block amide, and the like. In some instances, the regulator tubes are made from a metal.

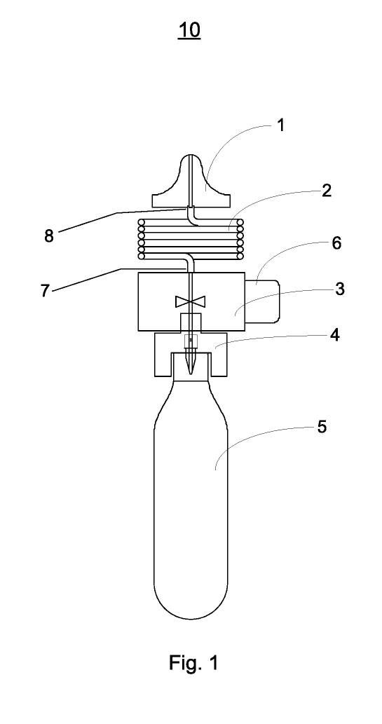

Referring to FIG. 1, an exemplary hand-held dispenser 10 with a regulator tube 2 having an inlet end 7 and an outlet end 8 and properties as described in the prior paragraphs is shown. The size of hand-held dispenser 10 is such that it may be easily held in a single hand. Hand-held dispenser 10 comprises a compressed gas cylinder 5 containing a therapeutic gas affixed to the bottom of a pierce pin block 4. Assembly of these two elements causes the pierce pin to rupture a thin metal cap on the head of the compressed gas cylinder 5 and, thus, allows the free flow of the compressed therapeutic gas through the pierce pin and into a stem valve 3. A porous filter is incorporated into the pierce pin block 4 to remove any particulates from the gas stream. The top of the pierce pin block 4 is coupled to the bottom of a stem valve 3. The stem valve 3 is an on/off valve that couples the compressed gas cylinder to the inlet end 7 of the regulator tube so that it prevents any downstream gas flow when the valve is closed and permits the free flow of gas when it is open. That is, the stem valve 3 typically allows the selective discharge of gas from the compressed gas cylinder through the regulator tube 2. Here the stem may be fastened to a ball valve, poppet valve, and the like. The valve is normally closed and in this variation, caused to open by manual depression of the on/off pushbutton 6. The top of the stem valve 3 is coupled to one end of a regulator tube 2. When the stem valve 3 is open, gas flows through the stem valve 3 and into the regulator tube 2. The other end of the regulator tube 2 is coupled to a nosepiece 1. The regulator tube 2 down-regulates the gas pressure and the flow rate to the desired value as the gas transits the tube, finally exiting through nosepiece 1. Here, the regulator tube 2 is coiled to minimize the overall size of the dispensing device. Other tube configurations may be used to fit the proposed device geometry and volume constraints.

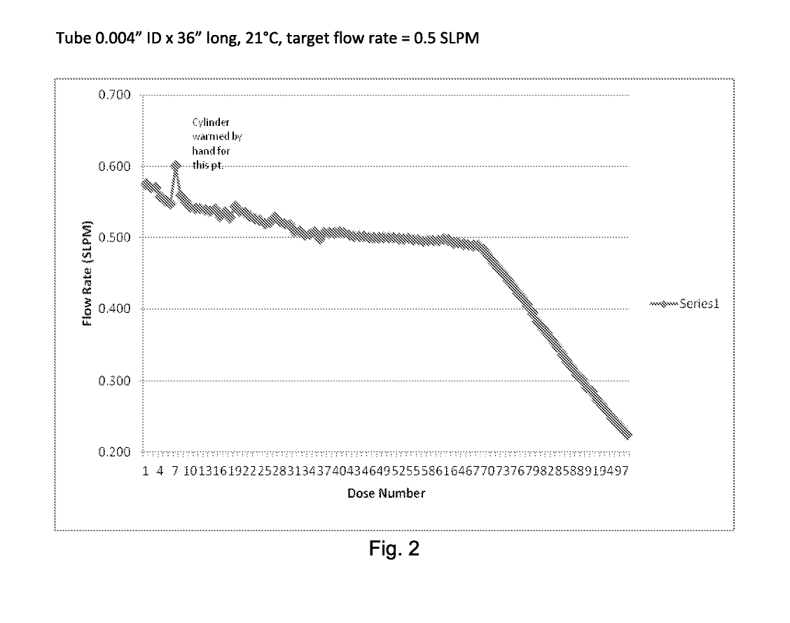

In one variation, a regulator tube having the dimensions of 0.004″ ID×36″ long (0.0101 cm ID×91.4 cm long) is employed in the hand-held intranasal delivery device. The flow rate performance of this regulator tube is shown in FIG. 2. Here the targeted flow rate was 0.5 standard liters per minute (SLPM). Data was collected for a temperature of 21° centigrade and an initial gas pressure in the compressed gas cylinder of 850 psi. The compressed carbon dioxide was delivered from a conventional miniature cylinder (12 grams CO₂). The data indicates that the flow rate is reasonably constant relative to the target of 0.5 SLPM for doses numbering 1 through approximately 67. This region of relative constant flow rate is termed the operating region. After dose 67, the pressure of the compressed gas is insufficient to maintain the target flow rate. This region of the operation is considered the gas depletion region.

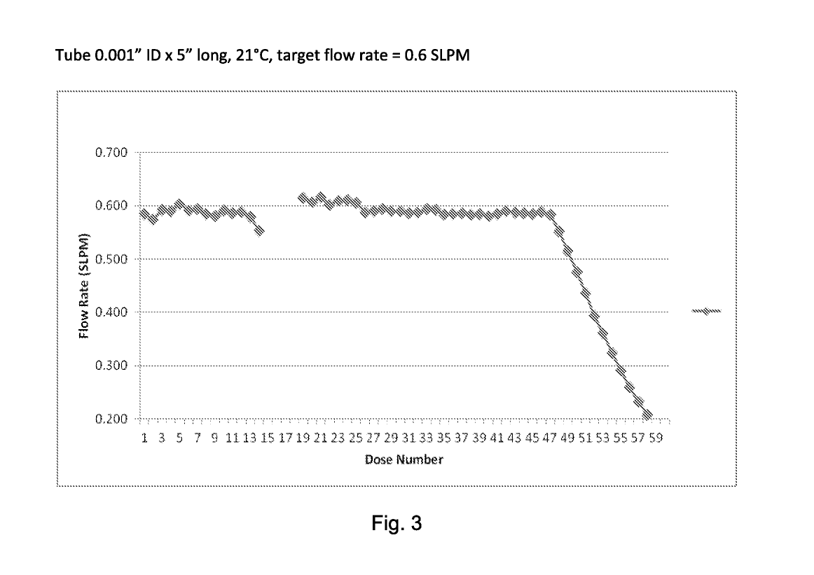

In another variation, a regulator tube having the dimensions of 0.001″ ID×5″ long (0.003 cm×12.7 cm long) is used in the hand-held intranasal delivery device. The flow rate performance of this tube is shown in FIG. 3. Here the targeted flow rate is 0.6 standard liters per minute (SLPM). Data was collected for a temperature of 21° centigrade and an initial gas pressure in the compressed gas cylinder of 850 psi. The data indicates that the flow rate is reasonably constant relative to the target of 0.6 SLPM for doses numbering 1 through approximately 46.

Methods for the intranasal delivery of therapeutic gases to a user are also described herein. In some variations, the method comprises the steps of obtaining a hand-held dispenser as described above, opening a stem valve and allow gas to flow from a compressed gas cylinder through the stem valve to a regulator tube, reducing and regulating gas flow in the regulator tube, and allowing the reduced and regulated gas flow to flow to a nosepiece.

In other variations, the method for intranasally delivering a therapeutic gas to a user includes inserting a hand-held dispenser including a regulator tube into a nostril of a user, and actuating a valve to selectively discharge the therapeutic gas from the compressed gas cylinder through the regulator tube and the nosepiece. Here the dispenser comprises a regulator tube having an inlet end, an outlet end, and a length therebetween, and also an internal diameter; a compressed gas cylinder comprising a therapeutic gas; a valve that couples the gas cylinder to the inlet end of the regulator tube; and a nosepiece coupled to the outlet end of the regulator tube. The regulator tube regulates the flow and pressure of the therapeutic gas released from the compressed gas cylinder.

The methods for delivering a therapeutic gas described above may be used to treat various medical conditions. For example, the therapeutic gases may be delivered to treat symptoms associated with headache (e.g., migraine headaches, tension-type headaches, cluster headaches), jaw pain, facial pain (e.g., trigeminal neuralgia), allergic conditions (e.g., rhinitis, including seasonal allergic rhinitis and perennial allergic rhinitis, and conjunctivitis), asthma, nervous disorders (e.g., epilepsy, Parkinson's disease), ischemic heart disease and pulmonary hypertension. Some variations of the method employ carbon dioxide as the delivered gas to treat seasonal allergic rhinitis or perennial allergic rhinitis. In another variation, nitric oxide or its variants is delivered to treat ischemic heart disease. In yet further variations, nitric oxide or its variants is delivered to treat pulmonary hypertension.

1. A hand-held dispenser comprising:
a regulator tube having an inlet end, an outlet end, and a length therebetween, and further having an internal diameter;
a compressed gas cylinder comprising a therapeutic gas;
a valve coupled to the gas cylinder and the inlet end of the regulator tube; and
a nosepiece coupled to the outlet end of the regulator tube.

2. The hand-held dispenser of claim 1, wherein the valve is a stem valve.

3. The hand-held dispenser of claim 1, wherein the therapeutic gas comprises carbon dioxide.

4. The hand-held dispenser of claim 1, wherein the regulator tube is coiled.

5. The hand-held dispenser of claim 1, wherein the length of the regulator tube ranges from about 2.54 cm (1.0 inch) to about 152.4 cm (60 inches).

6. The hand-held dispenser of claim 5, wherein the length of the regulator tube is about 91.4 cm (36 inches).

7. The hand-held dispenser of claim 1, wherein the length of the regulator tube ranges from about 12.7 cm (5.0 inches) to about 38.1 cm (15 inches).

8. The hand-held dispenser of claim 7, wherein the length of the regulator tube is about 12.7 cm (5 inches).

9. The hand-held dispenser of claim 1, wherein the internal diameter of the regulator tube ranges from about 0.00254 cm (0.001 inches) to about 0.0127 cm (0.005 inches).

10. The hand-held dispenser of claim 9, wherein the internal diameter of the regulator tube is about 0.003 cm (0.001 inches).

11. The hand-held dispenser of claim 9, wherein the internal diameter of the regulator tube is about 0.0102 cm (0.004 inches).

12. The hand-held dispenser of claim 1, wherein the ratio of the length to internal diameter of the regulator tube ranges from about 500:1 to about 12,000:1.

13. The hand-held dispenser of claim 12, wherein the ratio of the length to internal diameter of the regulator tube is about 5000:1.

14. The hand-held dispenser of claim 12, wherein the ratio of the length to internal diameter of the regulator tube is about 9000:1.

15. A method for intranasally delivering a therapeutic gas to user comprising:
inserting a hand-held dispenser into a nostril of a user, the dispenser comprising
a regulator tube having an inlet end, an outlet end, and a length therebetween, and further having an internal diameter;
a compressed gas cylinder comprising a therapeutic gas;
a valve coupled to the gas cylinder and the inlet end of the regulator tube; and
a nosepiece coupled to the outlet end of the regulator tube; and
actuating the valve to selectively discharge the therapeutic gas from the compressed gas cylinder through the regulator tube and the nosepiece,
wherein the regulator tube regulates the flow and pressure of the therapeutic gas released from the compressed gas cylinder.

16. The method of claim 15, wherein the therapeutic gas comprises carbon dioxide.

17. The method of claim 15, wherein the pressure of the therapeutic gas at the inlet end of the regulator tube ranges from about 300 psi to about 1800 psi.

18. The method of claim 17, wherein the pressure of the therapeutic gas at the inlet end of the regulator tube ranges from about 600 psi to about 1200 psi.

19. The method of claim 18, wherein the pressure of the therapeutic gas at the inlet end of the regulator tube ranges from about 750 psi to about 900 psi.

20. The method of claim 15, wherein the pressure of the therapeutic gas at the inlet end of the regulator tube is about 850 psi.

21. The method of claim 15, wherein the pressure of the therapeutic gas at the outlet end of the regulator tube is about 14.7 psi.

22. The method of claim 15, wherein the regulator tube delivers the therapeutic gas at a flow rate ranging from about 0.10 SLPM to about 1.0 SLPM.

23. The method of claim 22, wherein the regulator tube delivers the therapeutic gas at a flow rate of about 0.50 SLPM.

24. The method of claim 15, wherein the therapeutic gas is delivered to treat a medical condition.

25. The method of claim 24, wherein the medical condition is selected from the group consisting of headache, jaw pain, facial pain, allergic conditions, asthma, and nervous disorders.

26. The method of claim 25, wherein the medical condition is headache.

27. The method of claim 25, wherein the medical condition is an allergic condition.

28. The method of claim 27, wherein the allergic condition is an allergic rhinitis.

29. The method of claim 28, wherein the allergic rhinitis is seasonal allergic rhinitis.

30. The method of claim 28, wherein the allergic rhinitis is perennial allergic rhinitis.
